(19) United States
(12) Patent Application Publication
(10) Pub. No.: US 2011/0195927 A1
(43) Pub. Date: Aug. 11, 2011

(54) Compositions Comprising Polymers Having Amino Sugar Units and Methods of Making and Using Same

(75) Inventors: Erning Xia, Penfield, NY (US); Craig M. Cody, Scottsville, NY (US); Tammy J. Kleiber, Rochester, NY (US); Susan E. Burke, Batavia, NY (US)

(73) Assignee: COMPOSITIONS COMPRISING POLYMERS HAVING AMINIO SUGAR UNITES AND METHODS OF MAKING AND USING SAME

(21) Appl. No.: 13/062,402

(22) PCT Filed: Sep. 10, 2009
(86) PCT No.: PCT/US09/56440

§ 371 Date: Apr. 25, 2011

(30) Foreign Application Priority Data
Sep. 15, 2008 (US) 61096917

(51) Int. Cl. A61K 31/726; A61P 27/02; A61P 27/04

(52) U.S. Cl. 514/54

(57) ABSTRACT

Compositions comprise a polymer having units of an amino sugar, a polyol other than sorbitol, and boric acid, a salt thereof, or a combination thereof. Such compositions remain for an extended period in the eye and can be used for relief, treatment, or control of a condition of dry eye or discomfort resulting therefrom.

### CROSS REFERENCE

This application is a national-stage application, filed under 35 U.S.C. §371, of International Application PCT/US2009/056440, filed on Sep. 10, 2009, and claims the right of priority based on said International Application, which claims the right of priority based on U.S. Provisional Patent Application No. 61/096,917, filed on Sep. 15, 2008. Both International Application PCT/US2009/056440 and U.S. Provisional Patent Application No. 61/096,917 are incorporated herein by reference.

### BACKGROUND

The present invention relates to compositions comprising polymers that have amino sugar units, and methods of making and using such compositions. In particular, the present invention relates to such compositions and methods for treating or controlling condition of dry eye or discomfort resulting therefrom.

Dry eye, also known as keratoconjunctivitis sicca or dyslacrima, is a common ophthalmological disorder affecting millions of people. A patient with dry eye may experience burning, a feeling of dryness, and persistent irritation. In severe cases, dry eye can seriously impair a person's vision and hence handicap the sufferer in activities such as driving. Certain diseases such as Sjogren's disease manifest dry eye symptoms. Also, as people age, the lacrimal glands in the eye may produce less moisture, resulting in eyes that become dry, inflamed, itchy, and gritty.

Although it appears that dry eye may result from a variety of unrelated pathogenic causes, all presentations of the condition share a common feature, namely the breakdown of the precorneal tear film, which breakdown commonly results in dehydration of the exposed outer ocular surface and hence the symptoms described above.

A number of approaches exist for the treatment of dry eye. One common approach has been to supplement the ocular tear film using artificial tears instilled throughout the day. Examples of the tear substitute approach include the use of buffered, isotonic saline solutions and aqueous solutions containing water-soluble polymers that render the solutions more viscous and thus less easily shed by the eye by the washing action of the tear fluid. See, for example, U.S. Pat. No. 5,209,927 to Gressel et al.; U.S. Pat. No. 5,294,607 to Glonek et al.; and U.S. Pat. No. 4,409,205 to Shively.

Although these approaches have met with some success in some cases, significant challenges in the treatment of dry eye nevertheless remain. Such challenges include the fact that the use of tear substitutes, while temporarily effective, generally requires repeated application over the course of a patient's waking hours, not uncommonly ten or more times over the course of a day. Such an approach is inconvenient to a patient. Although increasing the viscosity of the dry-eye product may extend the duration of the product in the eye, there are practical challenges to formulate compositions having increased viscosity. For example, in order to achieve a desired high viscosity and effectively to extend the residence time of ophthalmic compositions in the eye, the proportion of a water-soluble polymer included in such compositions may have to be significantly increased, possibly presenting difficulties in the process of making, or dispensing, such compositions. Such compositions having very high concentrations of polymers also may be undesirable because they feel sticky in the eye or tend to blur vision.

Increases in the duration of the product, without the aforementioned disadvantages, would be highly desirable.

Some prior-art compositions include cellulose derivatives (e.g., carboxymethyl cellulose or hydroxypropyl cellulose), poly(acrylic acid), alginate, derivatives thereof, or pharmaceutically acceptable salts thereof as viscosity-enhancing agents. Alginate is a polysaccharide that comprises monomeric units of β-D-mannuronic acid and α-L-guluronic acid, or salts thereof, or derivatives of such acids or salts,

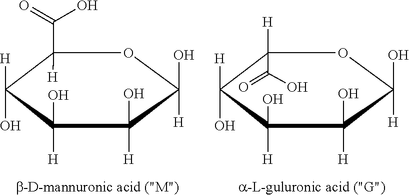

Some alginate polymers are block copolymers with blocks of the guluronic acid (or a salt thereof) monomeric units alternating with blocks of the mannuronic acid (or a salt thereof) monomeric units. Other alginate molecules have alternating single monomeric units of guluronic acid (or a salt thereof) and mannuronic acid (or a salt thereof). The ratio and distribution of the M and G components along with the average molecular weight affect the physical and chemical properties of the copolymer. See A. Haug et al., Acta Chem Scand, Vol. 20, 183-190 (1966). Alginate polymers have viscoelastic rheological properties and other properties that make it suitable for some medical applications. See G. Klock et al., “Biocompatibility of Mannuronic Acid-Rich Alginates,” Biomaterials, Vol. 18, No. 10, 707-713 (1997).

The use of alginate as a thickener for topical ophthalmic use is disclosed in U.S. Pat. No. 6,528,465 and U.S. Patent Application Publication 2003/0232089. U.S. Pat. No. 5,776,445 discloses the use of alginate as a drug delivery agent that is topically applied to the eye. Particularly, the amount of guluronic acid in the alginate was taught to exceed 50%.

U.S. Patent Application Publication 2003/0232089 teaches a dry-eye formulation that contains two polymer ingredients including alginate.

Ophthalmic compositions also can include other ingredients that provide additional properties. For example, polyols (e.g., glycerin) are known as demulcents and tonicity adjusting agents in ophthalmic formulations including formulations for the delivery of an active pharmaceutical agent. See; e.g., U.S. Pat. Nos. 5,075,104 and 5,209,927, which teach the use of a polyol with a carbomer polymer.

Therefore, in view of the shortcomings of prior-art compositions, there is a continued need to provide improved compositions that would remain on or in the eye over an extended period. In addition, it is also very desirable to provide improved compositions and methods for the treatment or control of the dry eye condition or discomfort resulting therefrom. It is also desirable to provide such compositions that are gentle to the ocular surface.

### SUMMARY

In general, the present invention provides a composition that comprises a polymer comprising units of amino sugar, and methods of preparing and using such composition.

In one aspect, the present invention provides a composition capable of treating or controlling a condition of dry eye or discomfort resulting therefrom.

In another aspect, the composition remains on or in the eye for an extended period of time. In one embodiment, such period of time is about two hours or longer.

In still another aspect, the composition is gentle to the ocular surface.

In yet another aspect, a composition of the present invention comprises: (a) a polymer comprising units of an amino sugar; (b) a polyol other than sorbitol; and (c) boric acid, a borate salt, or both.

In a further aspect, the polyol has 2 to 6 carbon atoms, such as 2, 3, 4, 5, or 6 carbon atoms, provided that when the polyol has six carbon atoms, it is other than sorbitol.

In still another aspect, the polyol has two hydroxyl groups.

In yet another aspect, the polyol comprises glycerin, propylene glycol, or both.

In yet another aspect, the polymer is selected from the group consisting of hyaluronic acid, chitosan, chitin, heparin, heparan, dermatan, chondroitin, copolymers thereof, and pharmaceutically acceptable salts thereof.

In a further aspect, the polymer comprises hyaluronic acid or a pharmaceutically acceptable salt thereof.

In a further aspect, the present invention also provides a method of treating or controlling a condition of dry eye or discomfort resulting therefrom. The method comprises administering to an eye of a subject suffering from such a condition any one of the compositions herein generally or specifically disclosed.

In still another aspect, such a composition comprises a solution, a dispersion, an emulsion (such as oil-in-water emulsion), a gelable composition, or a gel.

In yet another aspect, the present invention provides a method for preparing a pharmaceutical composition. The method comprises combining a polymer that comprises units of an amino sugar, a polyol other than sorbitol, and a material selected from the group consisting of boric acid, salts thereof, and combinations thereof.

Other features and advantages of the present invention will become apparent from the following detailed description and claims.

### DETAILED DESCRIPTION

In general, the present invention provides a composition that comprises a polymer comprising units of amino sugar, and methods of preparing and using such composition.

In one aspect, the present invention provides a composition and a method for treating or controlling a dry eye condition or discomfort resulting therefrom.

As used herein, the term “control” or grammatical derivatives thereof also include ameliorating, reducing, and preventing.

In another aspect, the composition remains on or in the eye for an extended period of time. In one embodiment, such period of time is about two hours or longer. In another embodiment, such period of time is about three, four, five, six, seven, or eight hours, or longer.

In still another aspect, the composition is gentle to the ocular surface.

In yet another aspect, a composition of the present invention comprises: (a) a polymer comprising units of an amino sugar; (b) a polyol other than sorbitol; and (c) boric acid, a borate salt, or both.

In a further aspect, the amino sugar is selected from the group consisting of compounds having Formula (I) or (II), and combinations thereof,

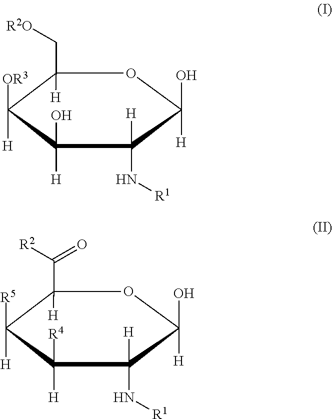

wherein R¹ is H, C(O)R⁶, or S(O)₂OH; R² is H or S(O)₂OH; R³ is H, C(O)OH, S(O)₂OH, or C(O)R⁶; R⁴ and R⁵ are independently C(O)OH, OR², S(O)₂OH, or C(O)R⁶; and wherein R⁶ is an alkyl group having 1-5 carbon atoms. In one embodiment, R⁶ is an alkyl group having 1-3 carbon atoms. In another embodiment, R⁶ is the methyl group. In still another embodiment, R⁶ is the ethyl group.

In still another aspect, the polymer is selected from the group consisting of hyaluronic acid, chitosan, chitin, heparin, heparan, dermatan, chondroitin, copolymers thereof, derivatives thereof, and pharmaceutically acceptable salts thereof.

In still another aspect, the polymer is a copolymer comprising units of said amino sugars and units of another sugar selected from the group consisting of glucose, mannose, galactose, combinations thereof, and derivatives thereof.

In yet another aspect, said derivatives of another sugar are selected from the group consisting of glucuronic acid, guluronic acid, iduronic acid, mannuronic acid, and combinations thereof.

In one embodiment, the polymer comprises linear chains, each comprising said amino sugars and at least one of said other sugar units. In another embodiment, the polymer comprises branched chains, each comprising said amino sugars and at least one of said other sugar units. In still another embodiment, the polymer comprising linear chains or branched chains that are cross-linked.

In another aspect, the mass average molecular weight of the polymer is in the range from about 5 kDa to about 20,000 kDa. Alternatively, the mass average molecular weight of the polymer is in the range from about 10 kDa to about 10,000 kDa, or from about 20 kDa to about 5,000 kDa, or from about 20 kDa to about 1,000 kDa, or from about 20 kDa to about 500 kDa, or from about 20 kDa to about 200 kDa, or from about 50 kDa to about 1,000 kDa, or from about 50 kDa to about 500 kDa, or from about 50 kDa to about 200 kDa, or from about 50 kDa to about 100 kDa.

In still another aspect, the polymer comprising units of amino sugars is present in a composition of the present invention at a concentration from about 0.001 to about 10 percent by weight of the total composition. Alternatively, the polymer comprising units of amino sugars is present in a composition of the present invention at a concentration from about 0.001 to about 5 percent, or from about 0.001 to about 3, or from about 0.001 to about 2, or from about 0.001 to about 1, or from about 0.001 to about 0.5, or from about 0.01 to about 5, or from about 0.01 to about 3, or from about 0.01 to about 2, or from about 0.01 to about 1, or from about 0.01 to about 0.5, or from about 0.1 to about 0.5, percent by weight of the total composition.

In still another aspect, the composition further comprises a synthetic carboxy-containing polymer. Non-limiting examples of such synthetic carboxy-containing polymers include poly(acrylic acid), poly(methacrylic acid), poly(crotonic acid), poly(itaconic acid), and copolymers thereof. In some embodiments, such synthetic carboxy-containing polymers can contain amino groups (e.g., polymers containing some units of amino-itaconic acid).

In yet another aspect, the synthetic carboxy-containing polymer is present in a composition of the present invention at a concentration from about 0.001 to about 5 percent, or from about 0.001 to about 3, or from about 0.001 to about 2, or from about 0.001 to about 1, or from about 0.001 to about 0.5, or from about 0.01 to about 5, or from about 0.01 to about 3, or from about 0.01 to about 2, or from about 0.01 to about 1, or from about 0.01 to about 0.5, or from about 0.1 to about 0.5, percent by weight of the total composition.

In a further aspect, the polyol has 2 to 6 carbon atoms, such as 2, 3, 4, 5, or 6 carbon atoms, provided that when the polyol has six carbon atoms, it is other than sorbitol. Non-limiting examples of suitable polyols include glycerin, ethylene glycol, propylene glycol, mannitol, xylitol, and combinations thereof. In one embodiment, the polyol is selected from the group consisting of glycerin, ethylene glycol, propylene glycol, butanediols, butanetriols, xylitol, pentanediols, pentanetriols, pentanetetraols, mannitol, and combinations thereof. In another embodiment, the polyol has 2-4 carbon atoms. In still another embodiment, the polyol has 2-3 carbon atoms.

In still another aspect, the polyol comprises two hydroxyl groups. In one embodiment, the polyol has two hydroxyl groups. In another embodiment, the polyol has three hydroxyl groups. In still another embodiment, the polyol is propylene glycol. In yet another embodiment, the polyol is glycerin. In a further embodiment, the polyol comprises two or more polyols. In yet another embodiment, the polyol is a combination of glycerin and propylene glycol.

In still another aspect, the amount of polyol or polyols in a composition of the present invention is in the range from about 0.001 to about 7 percent by weight of the total composition. Alternatively, the amount of polyol or polyols in a composition of the present invention is in the range from about 0.001 to about 5 percent, or from about 0.001 to about 3, or from about 0.001 to about 2, or from about 0.001 to about 1, or from about 0.001 to about 0.5, or from about 0.01 to about 5, or from about 0.01 to about 3, or from about 0.01 to about 2, or from about 0.01 to about 1, or from about 0.01 to about 0.5, or from about 0.1 to about 0.5, percent by weight of the total composition.

In yet another aspect, the amount of boric acid or salts thereof, or a combination thereof is in the range from about 0.001 to about 5 percent by weight of the total composition. Alternatively, the amount of boric acid or salts thereof, or a combination thereof is in the range from about 0.001 to about 3, or from about 0.001 to about 2, from about 0.001 to about 1, from about 0.01 to about 3, from about 0.01 to about 2, from about 0.01 to about 1, percent by weight of the total composition.

In a further aspect, said salt of boric acid include sodium, calcium, magnesium salt, or combinations thereof.

In yet another aspect, a composition of the present invention is free of alexidine, chlorhexidine, parabens, benzalkonium chloride, polymeric quaternary ammonium compounds, and derivatives thereof.

In a further aspect, a composition of the present invention comprises a pharmaceutically acceptable preservative, for use in multidose applications. Non-limiting examples of preservatives include sorbic acid and/or salts thereof, alexidine, chlorhexidine, parabens, benzalkonium chloride, polymeric quaternary ammonium compounds (e.g., polyhexamethylene biguanide, polyquaternium-1, polyquaternium-10, etc.), hydrogen peroxide, compounds the generate hydrogen peroxide (such as urea hydrogen peroxide or perborate salts), stabilized chlorine dioxide complexes, and derivatives thereof. Ophthalmically acceptable preservatives are particularly suitable. When a preservative is included in a composition, it is present in an amount in the range from about 0.0001 to about 5 percent by weight of the total composition. The specific amount will be sufficient to provide preservative efficacy and will depend upon the particular preservative used. For example, quaternary ammonium compounds are typically present in an amount from about 0.001 to about 0.2 percent (preferably, from about 0.001 to about 0.1 percent) by weight of the total composition. Hydrogen peroxide or a source thereof may be present in an amount from about 0.001 to about 3 percent (preferably, from about 0.001 to about 0.3 percent) by weight of the total composition. Stabilized chloride dioxide may be present in an amount from about 0.005 to about 0.2 percent by weight of the total composition.

The aqueous solutions employed in this invention may contain one or more additional ingredients that are commonly present in ophthalmic solutions, for example, tonicity-adjusting agents, buffers, antioxidants, viscosity-adjusting agents, surfactants, stabilizers, chelating agents, combinations thereof, and the like, which aid in making ophthalmic compositions more comfortable to the user.

In one aspect, a composition of the present invention can be adjusted with tonicity-adjusting agents to approximate the tonicity of normal lacrimal fluids that is equivalent to a 0.9 percent (by weight) solution of sodium chloride or a 2.8 percent (by weight) of glycerin solution. Alternatively, the compositions of the present invention desirably have osmolality in a range from about 180 mOsm/kg to about 400 mOsm/kg. Alternatively, the osmolality is in the range from about 180 mOsm/kg to about 320 mOsm/kg, (or from 200 to about 360 mOsm/kg, or from about 200 to about 320 mOsm/kg, or from about 220 to about 320 mOsm/kg, or from about 240 to about 280 mOsm/kg, or from about 220 to about 280 mOsm/kg, or from about 220 to about 260 mOsm/kg).

In another aspect, a composition of the present invention can comprise a buffering agent or system. Suitable buffers for use in compositions of the present invention include Good's buffers. Non-limiting examples of buffering agents include MES (2-(N-morpholino)ethanesulfonic acid hemisodium salt) having pKa of 6.1 at 25° C. and pH in the range of about 5.5-6.7; HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; BIS-TRIS (bis(2-hydroxyethyl)amino-tris(hydroxymethyl)methane) having pKa of 6.5 at 25° C. and pH in the range of about 5.8-7.2; citrate buffer (pH in the range of about 5.5-7.2); maleate buffer (pH in the range of about 5.5-7.2); succinate buffer (pH in the range of about 5.5-6.5); and malate buffer (pH in the range of about 4-6). Other pharmaceutically acceptable buffers that provide pH in the range of 5 to 7.5 also can be used.

A composition of the present invention can have a viscosity in the range from about 1 to about 50,000 centipoise (“cP”) or mPa·s (or alternatively, from about 2 to about 20,000, or from about 10 to about 10,000, or from about 10 to about 5,000, or from about 10 to about 1,000, or from about 10 to about 700, or from about 100 to about 1,000, or from about 100 to about 5,000, or from about 100 to about 10,000 or from about 500 to about 1,000, or from about 500 to about 5,000 cP or mPa·s).

The use of viscosity enhancing agents to provide the compositions of the invention with viscosities greater than the viscosity of simple aqueous solutions may be desirable to further increase the retention time in the eye. Such viscosity enhancing agents include, for example, polyvinyl alcohol, polyvinyl pyrrolidone, methyl cellulose, hydroxypropylmethyl cellulose, hydroxyethyl cellulose, carboxymethyl cellulose, hydroxypropyl cellulose or other agents known to those skilled in the art. Such agents are typically employed at a level of from about 0.01 to about 5 percent (alternatively, from about 0.1 to about 5 percent, or from about 0.1 to about 2 percent, or from about 0.1 to about 21 percent, or from about 0.1 to about 0.5 percent) by weight of the total composition.

Suitable surfactants include polyvinyl pyrrolidone, polyvinyl alcohol, polyethylene glycol, ethylene glycol, and propylene glycol. Other surfactants are polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij', Myr', and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). A surfactant helps a topical formulation to spread on the ocular surface.

Suitable antioxidants include, but are not limited to, ascorbic acid and its esters, sodium bisulfite, butylated hydroxytoluene, butylated hydroxyanisole, tocopherols, and combinations thereof. Antioxidants can be included in a composition of the present invention in an amount in the range from about 0.005 to about 0.05 percent by weight (or alternatively, from about 0.005 to about 0.02 percent, or from about 0.005 to about 0.01 percent, by weight) of the total composition.

The present invention also provides a method of ameliorating, reducing, treating, or preventing a condition of dry eye. The method comprises administering to an affected eye a composition that comprises: (a) a polymer comprising units of an amino sugar; (b) a polyol other than sorbitol; and (c) boric acid, a borate salt, or both.

Ingredients suitable for used in a method of the present invention are disclosed hereinabove.

In one aspect a composition for used in a method of the present invention further comprises a pharmaceutically acceptable carrier. In one embodiment, the composition comprises an aqueous solution. In another embodiment, the composition comprises an aqueous solution of which a viscosity increases upon being administered into an eye of a patient.

In another aspect, the composition has a pH in a range from about 5 to about 7.5. In one embodiment, the composition has a pH in the range from about 5.5 to about 7.5. In another embodiment, the composition has a pH in the range from about 6 to about 7.5 (or alternatively, from about 6 to about 7, or from about 5.5 to about 7, or from about 5.5 to about 6.5).

In another aspect, the composition can be applied in one or more drops to an ocular surface once per day, twice per day, or three or more times per day, as needed.

In still another aspect, the method provides relief to an ocular discomfort resulting from a dry eye condition.

In still another aspect, the method provides long-lasting relief to an ocular discomfort resulting from a dry eye condition. In one embodiment, such long-lasting relief allows a patient to apply the composition every 2, 3, 4, 5, 6, 7, or 8 hours to the affected eye. In one embodiment, such long-lasting relief allows a patient to apply the composition every 2 or 4 hours to the affected eye.

In a further aspect, the present invention provides a method for producing a composition for treating or controlling a condition of dry eye or discomfort resulting therefrom. The method comprises combining: (1) polymer comprising units of an amino sugar; (2) a polyol other than sorbitol; and (3) boric acid, a borate salt, or a combination thereof, to form the composition.

In yet another aspect, the method further comprises adding a preservative in a desired amount to the composition.

In still another aspect, the method further comprises adjusting a pH of the composition to a value in a range from about 5 to about 8 (or alternatively, from about 5 to 7.5, or from about 5.5 to 7.5, or from about 5 to 6.5, or from about 5.5 to 6.5, or from about 5.5 to 7) to produce a final composition.

In a further aspect, the method further comprises subjecting the mixture to a sterilization procedure. In one embodiment, the sterilization procedure can comprise exposing the mixture to α, β, or γ radiation; autoclaving the mixture; or heating the mixture to a temperature in arrange from about 100 to about 125° C., for 10 minutes or longer, but less than a time that would result in a degradation of the polymer comprising amino sugar units.

A composition of the present invention may be packaged in unit-dose (for single use) or multi-dose (for multiple use) containers.

Table 1 shows exemplary compositions of the present invention that were prepared and tested.

TABLE 1
Viscosity Increased Unexpectedly for Compositions of
Present Invention

| | Example | | | |
|---|---|---|---|---|
| Ingredient | 1 | 2 | 3 | 4 |
| Boric acid (wt. %) | 0.5 | 0.5 | 0.5 | 0.5 |
| Sodium borate | 0.014 | 0.014 | 0.014 | 0.014 |
| (wt. %) | | | | |
| Hyaluronic acid (wt. | 0.25 | 0 | 0.25 | 0.25 |
| %) | | | | |
| Glycerin (wt. %) | 0 | 1 | 1 | 1.5 |
| EDTA (wt. %) | 0.05 | 0.05 | 0.05 | 0.05 |
| Urea hydrogen | 0.1 | 0.1 | 0.1 | 0.1 |
| peroxide (wt. %) | | | | |
| Purified water | q.s. 100 | q.s. 100 | q.s. 100 | q.s. 100 |
| pH | 6.24 | 6.17 | 6.02 | 5.97 |
| Osmolality (mOsm/kg) | 116 | 217 | 227 | 277 |
| Viscosity (cps or | 4.48 | 1.49 | 65.5 | 80.9 |
| mPa · s) | | | | |

A comparison of Examples 1, 2, and 3 shows an unexpected and surprising result of the effect of the combination of hyaluronic acid (a polymer comprising units of amino sugar) and glycerin (a polyol) on the viscosity of the composition.

Table 2 shows another set of exemplary compositions of the present invention that were prepared and tested.

TABLE 2
Effect of Propylene Glycol and/or Glycerin

| | Example | | | |
|---|---|---|---|---|
| Ingredient | 1 | 5 | 6 | 7 |
| Boric acid (wt. %) | 0.5 | 0.5 | 0.5 | 0.5 |
| Sodium borate (wt. %) | 0.014 | 0.014 | 0.014 | 0.014 |
| Hyaluronic acid | 0.25 | 0.25 | 0.25 | 0.25 |
| (wt. %) | | | | |
| Glycerin (wt. %) | 0 | 0 | 1 | 1 |
| Propylene glycol | 0 | 0.6 | 0.6 | 0.6 |
| (wt. %) | | | | |
| EDTA (wt. %) | 0.05 | 0.05 | 0.05 | 0.05 |
| Urea hydrogen | 01. | 0.1 | 0.1 | 0.1 |
| peroxide (wt. %) | | | | |
| Purified water | q.s. 100 | q.s. 100 | q.s. 100 | q.s. 100 |
| pH | 6.24 | 6.18 | 6.03 | 5.95 |
| Osmolality (mOsm/kg) | 116 | 194 | 311 | 366 |
| Viscosity (cps or | 4.48 | 50.3 | 90.5 | 88.1 |
| mPa · s) | | | | |

A comparison of Examples 1 and 5, or 1 and 6, or 1 and 7 shows an unexpected and surprising result of the effect of the combination of hyaluronic acid (a polymer comprising units of amino sugar) and propylene glycol (a polyol), or of hyaluronic acid, glycerin, and propylene glycol on the viscosity of the composition.

Table 3 shows another set of exemplary compositions of the present invention that were prepared and tested.

TABLE 3
Effect of Boric Acid/Borate Salt

| | Example | | | | | |
|---|---|---|---|---|---|---|
| Ingredient | 8 | 9 | 10 | 11 | 12 | 13 |
| Boric acid | 0 | 0 | 0.5 | 0.5 | 0 | 0 |
| (wt. %) | | | | | | |
| Sodium borate | 0 | 0 | 0.014 | 0.014 | 0 | 0 |
| (wt. %) | | | | | | |
| Sodium | 0.6 | 0.6 | 0 | 0 | 0 | 0 |
| phosphate | | | | | | |
| monobasic | | | | | | |
| (wt. %) | | | | | | |
| Sodium | 0.3 | 0.3 | 0 | 0 | 0 | 0 |
| phosphate | | | | | | |
| dibasic (wt. %) | | | | | | |
| Sodium citrate | 0 | 0 | 0 | 0 | 1.5 | 1.5 |
| (wt %) | | | | | | |
| Citric acid | 0 | 0 | 0 | 0 | 0.045 | 0.045 |
| (wt. %) | | | | | | |
| Hyaluronic | 0.25 | 0.25 | 0.25 | 0.25 | 0.25 | 0.25 |
| acid | | | | | | |
| (wt. %) | | | | | | |
| Glycerin | 1 | 1 | 1 | 1 | 1 | 1 |
| (wt. %) | | | | | | |
| EDTA (wt. %) | 0.05 | 0 | 0.05 | 0 | 0.05 | 0 |
| Urea hydrogen | 0.1 | 0.1 | 0.1 | 0.1 | 0.1 | 0.1 |
| peroxide | | | | | | |
| (wt. %) | | | | | | |
| Purified water | q.s. 100 | q.s. 100 | q.s. 100 | q.s. 100 | q.s. 100 | q.s. 100 |
| pH | 6.51 | 6.52 | 6.03 | 6.53 | 6.44 | 6.48 |
| Osmolality | 281 | 276 | 226 | 223 | 302 | 307 |
| (mOsm/kg) | | | | | | |
| Viscosity | 93.7 | 98.4 | 570.9 | 945.8 | 57.5 | 78.7 |
| (cps or | | | | | | |
| mPa · s) | | | | | | |

A comparison of Examples 8, 10, and 12, or 9, 11, and 13 shows an unexpected and surprising result of the effect of the combination of hyaluronic acid (a polymer comprising units of amino sugar), glycerin (a polyol), and boric acid/borate on the viscosity of the composition.

Table 4 shows another set of exemplary compositions of the present invention that were prepared and tested.

TABLE 4
Effect of Amount of Polyol

| | Example | | | | | |
|---|---|---|---|---|---|---|
| Ingredient | 14 | 15 | 16 | 17 | 18 | 19 |
| Boric acid | 0.5 | 0.5 | 0.5 | 0.5 | 0.5 | 0.5 |
| (wt. %) | | | | | | |
| Sodium borate | 0.014 | 0.014 | 0.014 | 0.014 | 0.014 | 0.014 |
| (wt. %) | | | | | | |
| Hyaluronic | 0 | 0.15 | 0.15 | 0.25 | 0.25 | 0.25 |
| acid | | | | | | |
| (wt. %) | | | | | | |
| Propylene | 1 | 0.5 | 1 | 0.5 | 1 | 1.5 |
| glycol | | | | | | |
| (wt. %) | | | | | | |
| EDTA (wt. %) | 0.05 | 0.05 | 0.05 | 0.05 | 0.05 | 0.05 |
| Urea hydrogen | 0.1 | 0.1 | 0.1 | 0.1 | 0.1 | 0.1 |
| peroxide | | | | | | |
| (wt. %) | | | | | | |
| Purified water | q.s. 100 | q.s. 100 | q.s. 100 | q.s. 100 | q.s. 100 | q.s. 100 |
| pH | 6.35 | 6.30 | 6.25 | 6.25 | 6.22 | 6.21 |
| Osmolality | 241 | 181 | 249 | 182 | 253 | 322 |
| (mOsm/kg) | | | | | | |
| Viscosity | 1.1 | 121 | 124 | 532 | 549 | 566 |
| (cps or | | | | | | |
| mPa · s) | | | | | | |

Table 4 again shows an unexpected and surprising result of the effect of the combination of hyaluronic acid (a polymer comprising units of amino sugar), propylene glycol (a polyol), and boric acid/borate on the viscosity of the composition.

Table 5 shows another set of exemplary compositions of the present invention that were prepared and tested.

TABLE 5
Effect of Amount of Polyol

| | Example | | | | | |
|---|---|---|---|---|---|---|
| Ingredient | 20 | 21 | 22 | 23 | 24 | 25 |
| Boric acid | 0.5 | 0.5 | 0.5 | 0.5 | 0.5 | 0.5 |
| (wt. %) | | | | | | |
| Sodium borate | 0.014 | 0.014 | 0.014 | 0.014 | 0.014 | 0.014 |
| (wt. %) | | | | | | |
| Hyaluronic | 0 | 0.15 | 0.15 | 0.25 | 0.25 | 0.25 |
| acid | | | | | | |
| (wt. %) | | | | | | |
| Glycerin | 1 | 0.5 | 1 | 0.5 | 1 | 1.5 |
| (wt. %) | | | | | | |
| EDTA (wt. %) | 0.05 | 0.05 | 0.05 | 0.05 | 0.05 | 0.05 |
| Urea hydrogen | 0.1 | 0.1 | 0.1 | 0.1 | 0.1 | 0.1 |
| peroxide | | | | | | |
| (wt. %) | | | | | | |
| Purified water | q.s. 100 | q.s. 100 | q.s. 100 | q.s. 100 | q.s. 100 | q.s. 100 |
| pH | 6.17 | 6.18 | 6.11 | 6.16 | 6.06 | 5.97 |
| Osmolality | 215 | 169 | 225 | 173 | 229 | 286 |
| (mOsm/kg) | | | | | | |
| Viscosity | 1.0 | 136 | 141 | 502 | 547 | 547 |
| (cps or | | | | | | |
| mPa · s) | | | | | | |

Table 5 again shows an unexpected and surprising result of the effect of the combination of hyaluronic acid (a polymer comprising units of amino sugar), glycerin (a polyol), and boric acid/borate on the viscosity of the composition.

Table 6 shows another set of exemplary compositions of the present invention that were prepared and tested.

TABLE 6
Effect of Amount of Boric Acid/Borate Salt

| | Example | | | | | |
|---|---|---|---|---|---|---|
| Ingredient | 26 | 27 | 28 | 29 | 30 | 31 |
| Boric acid | 0.5 | 0.5 | 0.3 | 0.5 | 0.7 | 0.9 |
| (wt. %) | | | | | | |
| Sodium borate | 0.014 | 0.014 | 0.009 | 0.014 | 0.02 | 0.025 |
| (wt. %) | | | | | | |
| Hyaluronic | 0.25 | 0 | 0.25 | 0.25 | 0.25 | 0.25 |
| acid | | | | | | |
| (wt. %) | | | | | | |
| Glycerin | 0 | 1 | 1 | 0.5 | 1 | 1.5 |
| (wt. %) | | | | | | |
| EDTA (wt. %) | 0.05 | 0.05 | 0.05 | 0.05 | 0.05 | 0.05 |
| Urea hydrogen | 0.1 | 0.1 | 0.1 | 0.1 | 0.1 | 0.1 |
| peroxide | | | | | | |
| (wt. %) | | | | | | |
| Purified water | q.s. 100 | q.s. 100 | q.s. 100 | q.s. 100 | q.s. 100 | q.s. 100 |
| pH | 6.24 | 6.17 | 5.89 | 6.03 | 6.08 | 6.06 |
| Osmolality | 115 | 216 | 189 | 227 | 263 | 305 |
| (mOsm/kg) | | | | | | |
| Viscosity | 34.7 | 1.37 | 179.5 | 423.5 | 370.7 | 438.5 |
| (cps or | | | | | | |
| mPa · s) | | | | | | |

Table 6 again shows an unexpected and surprising result of the effect of the combination of hyaluronic acid (a polymer comprising units of amino sugar), glycerin (a polyol), and boric acid/borate on the viscosity of the composition (compare Examples 26, 27 to Examples 28, 29, 30, and 31). Viscosity of a combination of the three ingredients increased unexpectedly to a magnitude that the absence of either hyaluronic acid or polyol could not achieve.

Unexpectedly, when sorbitol was combined with hyaluronic acid and boric acid/borate, the viscosity of the composition decreased, as the concentration of sorbitol increased. Table 7 below shows the results of this testing.

TABLE 7
Effect of Sorbitol

| | Ingredient (wt. %) | | | | | |
|---|---|---|---|---|---|---|
| | Boric | Sodium | Hyaluronic | | | Viscosity |
| Example | acid | borate | acid | Sorbitol | pH | (cp) |
| 32 | 0.5 | 0.014 | 0.025 | 0 | 6.97 | 9.04 |
| 33 | 0.5 | 0.014 | 0.05 | 0 | 6.98 | 35.4 |
| 34 | 0.5 | 0.014 | 0.1 | 0 | 6.94 | 130.6 |
| 35 | 0.5 | 0.014 | 0.025 | 0.5 | 4.82 | 8 |
| 36 | 0.5 | 0.014 | 0.05 | 0.5 | 4.85 | 29.6 |
| 37 | 0.5 | 0.014 | 0.1 | 0.5 | 4.9 | 92.1 |
| 38 | 0.5 | 0.014 | 0.025 | 1 | 4.3 | 7.34 |
| 39 | 0.5 | 0.014 | 0.05 | 1 | 4.36 | 22 |
| 40 | 0.5 | 0.014 | 0.1 | 1 | 4.44 | 77 |

In another aspect, a composition of the present invention can be used as a vehicle for an ophthalmic active ingredient (such as an ophthalmic drug) to provide a medicament that remains in or on the eye for an extended period, such as 2, 3, 4, 5, 6, 7, 8 hours or longer.

In one embodiment, a composition for treating or controlling a condition of dry eye or discomfort resulting therefrom, the composition consists essentially of: (a) polymer that comprises units of an amino sugar, said polymer being present at a concentration from about 0.1 to about 0.5 percent by weight of the total composition; (b) a polyol at a concentration from about 0.01 to about 2 percent by weight of the total composition; (c) boric acid, a borate salt, or a combination thereof at a concentration from about 0.01 to about 1 percent by weight of the total composition; and (d) water; wherein the composition has a pH from about 5.5 to about 7.5. In another embodiment, the polymer comprises or consists essentially of hyaluronic acid. In still another embodiment, the polyol comprises or consists essentially of glycerin. In yet another embodiment, the polyol comprises or consists essentially of glycerin and propylene glycol.

In another aspect, any one of the compositions of the present invention can be formed into a solution, an emulsion (such as an oil-in-water emulsion), a dispersion, a getable composition, or a gel.

In a typical production of a composition of the present invention, a volume of purified water that is equivalent to from about 85 to about 90 percent of the total batch weight (the temperature of purified water should be below 40° C. before other ingredients are added) is added into a sterilized stainless steel mixing vessel equipped with a stirring mechanism. The polymer comprising units of an amino sugar is added slowly with continued stirring and mixed thereafter for at least 30 minutes. Other ingredients are added slowly to the vessel over a period of about 30 minutes or longer (e.g., up to about 2 hours). The contents of the vessel is further mixed for another 15 minutes or longer (e.g., up to about 2 hours), then sterilized by any well-known method applicable for sterilization of pharmaceutical compositions. The composition is ready for packaging, storage, and use.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising: (a) a polymer comprising units of an amino sugar; (b) a polyol other than sorbitol; and (c) boric acid, a salt thereof, or a combination thereof.

2. The composition of claim 1, wherein the amino sugar is selected from the group consisting of compounds having Formula (I) or (II), and combinations thereof. 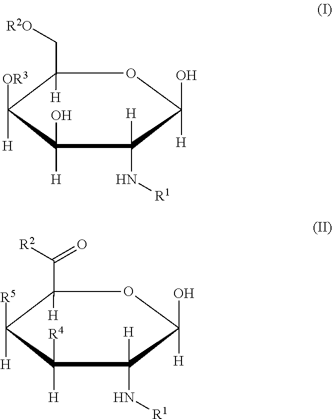
wherein R¹ is H, C(O)R⁶, or S(O)₂OH; R² is H or S(O)₂OH; R³ is H, C(O)OH, S(O)₂OH, or C(O)R⁶; R⁴ and R⁵ are independently C(O)OH, OR², S(O)₂OH, or C(O)R⁶; and wherein R⁶ is an alkyl group having 1-5 carbon atoms.

3. The composition of claim 2, wherein the polymer comprises a copolymer comprising units of said amino sugars and units of another sugar selected from the group consisting of glucose, mannose, galactose, combinations thereof, and derivatives thereof.

4. The composition of claim 2, wherein R⁶ is a methyl group.

5. The composition of claim 1, wherein the amino sugar is selected from the group consisting of hyaluronic acid, chitosan, chitin, heparin, heparan, dermatan, chondroitin, copolymers thereof, derivatives thereof, and pharmaceutically acceptable salts thereof.

6. The composition of claim 1, wherein the polymer is present in the composition in an amount in a range from about 0.001 to about 10 percent by weight of the total composition.

7. The composition of claim 1, wherein the polymer comprises hyaluronic acid or a pharmaceutically acceptable salt thereof.

8. The composition of claim 1, wherein the composition further comprises a synthetic carboxy-containing polymer.

9. The composition of claim 1, wherein the polyol has 2 to 6 carbon atoms.

10. The composition of claim 9, wherein the polyol comprises glycerin, propylene glycol, or a combination thereof.

11. The composition of claim 9, wherein the polyol is present in an amount from about 0.001 to about 7 percent by weight of the composition.

12. The composition of claim 1, further comprising a material selected from the group consisting of tonicity-adjusting agents, buffers, antioxidants, viscosity-enhancing agents, pH-adjusting agents, surfactants, stabilizers, chelating agents, and combinations thereof.

13. An ophthalmic composition consisting essentially of: (a) a polymer comprising units of an amino sugar; and (b) a polyol having 2-6 carbon atoms, other than sorbitol; (c) boric acid, a salt thereof, or a combination thereof; (d) a preservative; (e) a chelating agent; (f) a pH-adjusting agent; and (g) water; wherein the amino sugar is selected from the group consisting of compounds having Formula (I) or (II), and combinations thereof 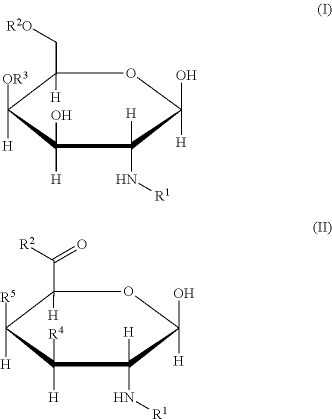
wherein R¹ is H, C(O)R⁶, or S(O)₂OH; R² is H or S(O)₂OH; R³ is H, C(O)OH, S(O)₂OH, or C(O)R⁶; R⁴ and R⁵ are independently C(O)OH, OR², S(O)₂OH, or C(O)R⁶; R⁶ is an alkyl group having 1-5 carbon atoms; the polymer is present in an amount from about 0.01 to about 3 percent by weight of the composition; the polyol is present in amount from about 0.01 to about 3 percent by weight of the composition; the composition has an osmolality in a range from about 180 to about 320 mOsm/kg.

14. An ophthalmic composition comprising: (a) a polymer comprising units of an amino sugar; and (b) a polyol having 2-6 carbon atoms, other than sorbitol; and (c) boric acid, a salt thereof, or a combination thereof; wherein the amino sugar is selected from the group consisting of compounds having Formula (I) or (II), and combinations thereof. 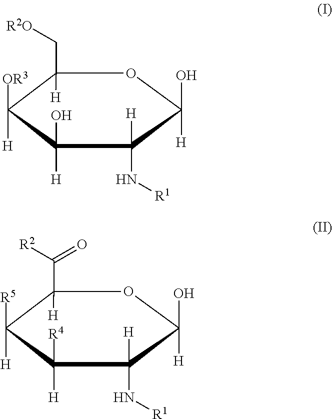
wherein R¹ is H, C(O)R⁶, or S(O)₂OH; R² is H or S(O)₂OH; R³ is H, C(O)OH, S(O)₂OH, or C(O)R⁶; R⁴ and R⁵ are independently C(O)OH, OR², S(O)₂OH, or C(O)R⁶; R⁶ is an alkyl group having 1-5 carbon atoms; the polymer is present in an amount from about 0.01 to about 3 percent by weight of the composition; the polyol is present in amount from about 0.01 to about 3 percent by weight of the composition; the composition has an osmolality in a range from about 180 to about 320 mOsm/kg.

15. A method for treating or controlling a condition of dry eye or discomfort resulting therefrom in a subject, the method comprising administering a composition into an affected eye of the subject; wherein the composition comprises (a) a polymer comprising units of an amino sugar; (b) a polyol other than sorbitol; and (c) boric acid, a salt thereof, or a combination thereof; wherein (i) the polymer, (ii) the polyol, and (iii) boric acid, a salt thereof, or a combination thereof, each present in an amount effective to relieve said dry eye condition or said discomfort.

16. The method of claim 15, wherein the amino sugar is selected from the group consisting of compounds having Formula (I) or (II), and combinations thereof. 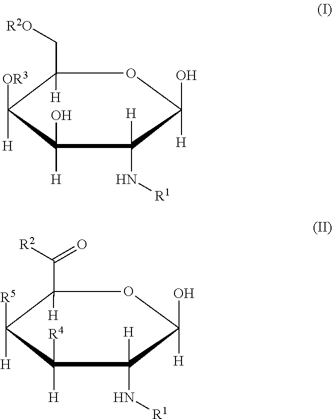
wherein R¹ is H, C(O)R⁶, or S(O)₂OH; R² is H or S(O)₂OH; R³ is H, C(O)OH, S(O)₂OH, or C(O)R⁶; R⁴ and R⁵ are independently C(O)OH, OR², S(O)₂OH, or C(O)R⁶; R⁶ is an alkyl group having 1-5 carbon atoms; the polymer is present in an amount from about 0.01 to about 3 percent by weight of the composition; the polyol is present in amount from about 0.01 to about 3 percent by weight of the composition; the composition has an osmolality in a range from about 180 to about 320 mOsm/kg.

17. The composition of claim 16, wherein the polymer is hyaluronic acid or a pharmaceutically acceptable salt thereof, and the polyol comprises glycerin, propylene glycol, or a combination thereof.

18. The method of claim 15, wherein said administering comprising instilling a drop of the composition in said affected eye every two hours or longer.

19. A method for preparing a composition, the method comprising combining: (a) a polymer comprising units of an amino sugar; (b) a polyol other than sorbitol; and (c) boric acid, a salt thereof, or a combination thereof.

20. The method of claim 19, wherein said combining further comprises adding a material selected from the group consisting of tonicity-adjusting agents, buffers, antioxidants, viscosity-enhancing agents, pH-adjusting agents, surfactants, stabilizers, chelating agents, and combinations thereof, to said composition.
